(19) United States
(12) Patent Application Publication
(10) Pub. No.: US 2001/0021387 A1
(43) Pub. Date: Sep. 13, 2001

(54) Hydrophobically modified cellulose ethers, their preparation and use

(75) Inventors: Ralf Krammer, Ginsheim-Gustavburg (DE); Reinhard Doenges, Bad Soden (DE); Juergen Kirchner, Wiesbaden (DE); Horst Wurm, Wiesbaden (DE)

Correspondence Address: CLARIANT CORPORATION, 4331 CHESAPEAKE DR, ATTN: INDUSTRIAL PROPERTY DEPT, CHARLOTTE, NC, 28216, US

(73) Assignee: Clariant GmbH

(21) Appl. No.: 09/784,534

(22) Filed: Feb. 15, 2001

(30) Foreign Application Priority Data
Feb. 15, 2000 (DE) 100 06 603.8

(51) Int. Cl. A61K 31/715; A61K 31/695; A61K 7/00; A61K 9/14; C07H 1/00; C07H 3/00

(52) U.S. Cl. 424/401; 536/84; 536/85; 536/90; 536/124; 514/57; 514/63; 424/488

(57) ABSTRACT

The present invention relates to water-soluble, organosilylated cellulose ethers in which the organosilyl groups are attached hydrolysis-stably to the cellulose ethers and the remaining substituents on the silicon atoms of the organosilyl groups are likewise stable to hydrolysis. The cellulose ethers of the invention exhibit a strongly thickening action in aqueous solution, even at very low degrees of silylation, and are suitable as thickeners, for example, in paints, adhesives, and cosmetics. They are further suitable as protective colloids in the preparation of polymer dispersions.

### FIELD OF THE INVENTION

The present invention relates to water-soluble, silylated cellulose ethers, to processes for preparing them, and to their use.

### BACKGROUND OF THE INVENTION

Cellulose ethers have a broad spectrum of industrial applications. They are used as thickeners, binders and dispersants, water retention agents, protective colloids, stabilizers, suspension agents, emulsifiers, and film formers.

In emulsion paints, cellulose ethers control the consistency and the water retention capacity, reduce the sedimentation of the pigments and fillers, and reinforce the adhesion and binding capacities. An important application-oriented objective in the preparation of emulsion paints is the setting of a rheology which as far as possible is Newtonian, since such systems have distinct advantages in respect of brushability, leveling and splashing over commercially customary emulsion paints, which have a pronounced pseudoplasticity. The desired rheology is achieved by using cellulose ethers which have been modified with long-chain alkyl groups, and/or by using conventional cellulose ethers in combination with synthetic thickeners having an associative action.

U.S. Pat. No. 4,228,277 describes modifying hydroxyethylcellulose by incorporating fewer long-chain alkyl groups, thereby changing the rheology in the manner described above and suppressing the splashing tendency.

As described in EP-A-0 476 507, the same effects are achieved with hydroxyethylcellulose modified by perfluoroalkyl substituents at degrees of substitution which are lower by a factor of from 10 to 100.

More advantageous, however, would be hydrophobically modified cellulose ethers which exhibit a comparable activity at comparably low degrees of substitution while being free from substituents containing fluorine.

### SUMMARY OF THE INVENTION

In the radical polymerization of water-insoluble vinyl monomers in aqueous, solvent-free reaction media, water-soluble cellulose derivatives are used as protective colloids, besides other polymeric carbohydrates, such as starch and dextrans, for example, since, after polymerization has taken place, it is necessary to stabilize the polymer in such systems. In the preparation of polyvinyl acetate dispersions by emulsion polymerization, for example, hydroxyethylcellulose is the most frequently used protective colloid. The choice of protective colloid exerts a significant influence over a large number of quality criteria of the polymer dispersion. These include the stability, the rheology, the viscosity, the size of the polymer particles, and the amount of coagulum, which is retained when the dispersion is filtered through a sieve.

The critical process in emulsion polymerization as far as the function of the protective colloid is concerned is the grafting of the monomer onto the colloid, the grafting rate depending on the one hand on the choice of initiator system and on the other hand on the nature of the protective colloid. With a low grafting rate it is necessary, accordingly, to increase the amount of protective colloid used, which results in an increased hydrophilicity of the polymer when drawn down to a film, as a result of which there is increased water absorption, which is unwanted. Moreover, an increase in the amount used is associated with an increase in costs. According to U.S. Pat. No. 4,845,175, the amount of protective colloid can be reduced if the protective colloid used comprises hydrophobically modified hydroxyethylcellulose.

It is known that introducing silyl groups into organic compounds results in a significant increase in the lipophilic character of the compounds thus modified (D. Klemm, B. Philipp, T. Heinze, U. Heinze, W. Wagenknecht, Comprehensive Cellulose Chemistry, Volume 2, Wiley-VCH Verlag, 1998, page 274).

Specific silicon compounds have already been used a number of times to modify cellulose ethers.

For instance, U.S. Pat. No. 4,474,950 uses special silanes in order to modify water-soluble cellulose ethers such that they can be stirred without lumps into an aqueous medium over a wide pH range.

U.S. Pat. No. 4,992,538 describes how, using certain organosilicon compounds, modified cellulose ethers form water-insoluble films if they are dried from an aqueous solution in the presence of atmospheric carbon dioxide. For this purpose it is necessary for at least one hydrolyzable group, such as a halogen atom, an alkoxy, aryloxy, acyloxy or siloxy group or an amino or thio function, to be attached to the silicon atom, since only in that case is it possible to develop the crosslinks which are the cause of film formation.

It is an object of the present invention to develop new kinds of hydrophobically modified, water-soluble cellulose ethers which exhibit a strongly thickening action in aqueous solution even at very low degrees of substitution with regard to the hydrophobic substituents.

### DESCRIPTION OF THE PREFERRED EMBODIMENTS

It has surprisingly now been found that water-soluble cellulose ethers silylated with at least one organosilyl group containing one or more silicon atoms, wherein the organosilyl group(s) is (are) attached hydrolysis-stably to the cellulose ethers and the remaining substituents of the silicon atom or silicon atoms of the organosilyl group(s) are stable to hydrolysis, exhibit a strongly thickening action in aqueous solution even at very low degrees of substitution.

The invention accordingly provides water-soluble cellulose ethers silylated with at least one organosilyl group containing one or more silicon atoms, wherein the organosilyl group(s) is (are) attached hydrolysis-stably to the cellulose ethers and the remaining substituents of the silicon atom or silicon atoms of the organosilyl group(s) are stable to hydrolysis.

The silyl-containing reagent or reagents for silylating the cellulose ethers preferably comprise organosilicon compounds having the compositions

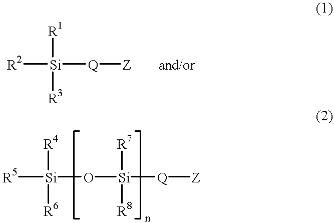

where

a) R¹ to R⁸ independently of one another are identical or different and are straight-chain or branched alkyl radicals, straight-chain or branched alkenyl radicals, aryl radicals and/or arylalkyl radicals containing straight-chain and/or branched alkyl groups,

b) Z is a reactive functional group suitable for covalent bonding to cellulose ethers and selected from the group consisting of Cl, Br, I, isocyanate, epoxy groups, glycidyloxy groups, acid halide groups and/or acid anhydride groups,

c) Q is a spacer group between the reactive group Z and the organosilyl group, and

d) n is from 1 to 100.

Preferably, R¹ to R⁸ comprise C₁-C₂₀ alkyl radicals or phenyl radicals. Suitable reactive functional groups Z are preferably epoxy groups. As the spacer group Q, C₁-C₂₀ hydrocarbon chains are preferably suitable. n is preferably from 1 to 50.

The average number of silyl groups per anhydroglucose unit (DS silyl) is preferably from 0.0002 to 0.2.

Cellulose ethers used with preference for the silylation are hydroxyethylcellulose, hydroxypropylcellulose, carboxymethylcellulose, carboxymethylhydroxyethylcellulose, methylcellulose, methylhydroxyethylcellulose and methylhydroxypropylcellulose.

The present invention also provides processes for preparing the silylated cellulose ethers of the invention.

In one process (A), cellulose is reacted with base catalysis with one or more etherifying agents selected from the group consisting of ethylene oxide, propylene oxide, methyl chloride, monochloroacetic acid and sodium monochloroacetate and at least one silylating reagent.

In one process (B) the starting material is a cellulose ether, which is reacted with base catalysis with at least one silylating reagent.

Cellulose ethers which are suitable with preference for the silylation are hydroxyethylcellulose, hydroxypropylcellulose, carboxymethylcellulose, carboxymethylhydroxyethylcellulose, methylcellulose, methylhydroxyethylcellulose or methylhydroxypropylcellulose. In both processes, suitable silylating reagents are preferably organosilanes of the compositions (1) and/or (2). Particular preference is given to organosilanes of the compositions (1) and/or (2) in which R¹ to R⁸ are C₁-C₂₀ alkyl radicals or phenyl radicals, Z is an epoxy group, and the spacer group Q is a C₁-C₂₀-hydrocarbon chain.

In both processes, the reactions take place preferably in a suspension medium. Preferred suspension media used are lower alcohols, such as isopropanol or tert-butanol for example, or ketones, such as acetone, for example. The weight ratio of suspension medium to cellulose or of suspension medium to cellulose ether is in both cases preferably from 3:1 to 30:1 and with particular preference from 8:1 to 15:1.

Bases used are, normally, aqueous solutions of alkali metal hydroxides, especially sodium hydroxide.

In the case of process (A), the base/anhydroglucose unit molar ratio is preferably from 1.0 to 1.5 and in process B it is preferably from 0.1 to 1.0. The water content of the reaction mixtures is preferably from 5 to 30 and with particular preference from 10 to 20 mol of water per anhydroglucose unit.

In the case of process (A), an advantageous procedure is to introduce the suspension medium initially, to add the cellulose and to render the batch alkaline using the aqueous base and then to homogenize it thoroughly and then to stir it without supplying heat, with cooling if appropriate, for preferably from 0.5 to 2 hours. Thereafter, the etherifying reagent and the silylating reagent are added together or in succession. A further possibility is to add the silylating reagent in the course of the reaction, under the reaction conditions described below. The batch is brought to the preferred temperature of from 40 to 120° C., in particular from 80 to 100° C., and is heated preferably for 2 to 6 hours. After cooling, it is neutralized with an acid, preferably hydrochloric acid, nitric acid or acetic acid, preferably to a pH of from 6 to 8. The suspension medium is removed by decanting or filtering, and the crude cellulose mixed ether may be freed from the adhering byproducts, such as polyglycols, glycol ethers and salts, for example, by extraction with aqueous alcohols or ketones having a preferred water fraction of from 10 to 50% by weight, especially isopropanol, ethanol or acetone. Drying under reduced pressure or atmospheric pressure and at from 50 to 120° C. gives the desired cellulose mixed ether as a colorless to pale yellowish powder.

In the case of process (B), the suspension medium is introduced initially, the cellulose ether is added and the batch is rendered alkaline using the aqueous base, homogenized thoroughly and stirred without supplying heat, with cooling if appropriate, for preferably from 0.5 to 2 hours. Thereafter, the silylating reagent is added and the batch is heated to the preferred temperature of from 40 to 120° C., in particular from 80 to 100C, and is heated preferably for 2 to 6 hours.

After cooling, it is neutralized with an acid, preferably hydrochloric acid, nitric acid or acetic acid, preferably to a pH of from 6 to 8. The suspension medium is subsequently removed by decanting or filtering, and the crude product may then be freed from the adhering byproducts and salts by extraction with aqueous alcohols or ketones having a preferred water fraction from 10 to 50% by weight, especially isopropanol, ethanol and acetone. Drying under reduced pressure or atmospheric pressure and at from 50 to 120° C. gives the desired cellulose mixed ether as a colorless to pale yellowish powder. If required, it is possible with both processes to adjust the desired degree of polymerization of the cellulose ether before or during its preparation process by adding a peroxo compound, such as hydrogen peroxide or a peroxodisulfate salt or another oxidizing agent, such as sodium chlorite, for example. The abovementioned methods of molecular weight reduction, and their respective technical implementation, are described in the prior art (T. M. Greenway in “Cellulosic Polymers, Blends and Composites”, edited by R. D. Gilbert, Carl Hanser Verlag Munich, 1994, p. 178 ff.).

Preferred reaction apparatus for preparing the silylated cellulose ethers of the invention are, for example, stirred vessels, mixers and extruders. In principle, however, it is possible to use any reaction apparatus which is customary for the preparation of cellulose derivatives and which ensures sufficient mixing of the cellulose or of the water-soluble cellulose ether with the silylating reagent.

The preparation of the epoxyorganosilanes which are used with preference for the silylation may take place, for example, by hydrosilylating allyl glycidyl ether with triorgano-H-silanes, with platinum catalysis, in toluene in accordance with a method of E. P. Plueddemann and G. Fanger, J. Am. Chem. Soc. 81 (1959), 2632.

Depending on the silylating reagent and on the degree of silylation, the application spectrum of the silylated cellulose ethers of the invention is diverse.

They are particularly suitable as auxiliaries with a thickening action in paints, adhesives and cosmetics, an advantageous feature being that the effect begins even at very low degrees of silylation.

The cellulose ethers of the invention are especially suitable for reducing the splashing tendency of emulsion paints. Advantageously, this is accompanied by only slight thickening of the paints, and the splashing behavior can be improved even at relatively low paint viscosities. At the same time, the paints exhibit good brushability and high abrasion resistance.

The invention additionally provides for the use of the cellulose ethers of the invention as protective colloids in the preparation of aqueous polymer dispersions. A preferred application in this context is the free-radically initiated polymerization of ethylenically unsaturated monomers in aqueous emulsion.

### EXAMPLES

The examples which follow serve to illustrate the invention without, however, restricting it.

### Example 1

Preparation of Inventive Cellulose Ethers by Silylation of Cellulose with Different Epoxyorganosilanes (Process (A))

In a 2 l glass reactor with anchor stirrer, finely ground cellulose is suspended in a solvent mixture (LM) of t-butanol and isopropanol. After the suspension has been rendered inert (by evacuation and flooding with nitrogen), a solution of sodium hydroxide and water is run in at 20° C. with stirring. The suspension is rendered alkaline at 20° C. for 60 minutes. Subsequently, ethylene oxide (EO) is run in and the temperature is held at 40° C. for 1 hour and then 80° C. for 90 minutes. Subsequently, the desired amount of the corresponding epoxyorganosilane, dissolved in t-butanol, is added at about 80° C. and etherification is carried out at 80° C. for 3 hours. After the product is cooled to room temperature, it is neutralized with approximately 65% strength nitric acid and glacial acetic acid. The product is filtered off with suction, washed with 80% strength aqueous acetone, digested in 100% strength acetone, and dried at 70° C.

For the batches a to f, Table 1 compiles the amounts of the reactants used, the degrees of substitution MS(OC₂H₄OH) and DS(organosilyl group), and the viscosities (2% strength solution, Hoeppler method) of the products. The epoxyorganosilanes used for the various batches are listed in Table 2. It is evident that the silylation brings about a marked increase in the viscosity.

TABLE 1
Batches a to f from Example 1

| | | |
|---|---|---|
| | Degrees of | |
| Amounts used (g) | substitution | Viscosities |

| Batch | Cellulose | LM | NaOH | EO | Epoxyorganosilane | MS | DS | (mPas) |
|---|---|---|---|---|---|---|---|---|
| a | 85 | 727 | 26 | 82 | — | 2.60 | — | 1780 |
| b | 85 | 727 | 26 | 82 | 24 | 2.67 | 0.030 | 14000 |
| c | 85 | 727 | 26 | 82 | 20 | 2.64 | 0.035 | 2771 |
| d | 85 | 727 | 26 | 82 | 15 | 2.68 | 0.0047 | 5860 |
| e | 85 | 727 | 26 | 82 | 10 | 2.75 | <0.0025 | 3290 |
| f | 85 | 727 | 26 | 82 | 14 | 2.75 | <0.0025 | 3408 |

TABLE 2
Epoxyorganosilanes used as silylating reagents in Example 1

| Batch | Silylating reagent |
|---|---|
| b | 3-(2,3-epoxypropoxy)propyltriethylsilane |
| c | 3-(2,3-epoxypropoxy)propylphenyldimethylsilane |
| d | 3-(2,3-epoxypropoxy)propyltri-n-hexylsilane |
| e | 3-(2,3-epoxypropoxy)propyloctadecyldimethylsilane |
| f | 3-(2,3-epoxypropoxy)propyloctadecyldimethylsilane |

### Example 2

Preparation of an Inventive Cellulose Ether by Silylation of hydroxyethylcellulose with 3-(2,3-epoxypropoxy)propylphenyldimethylsilane (Process (B))

In a 1 I three-necked flask with stirrer, 90 g of hydroxyethylcellulose (®Tylose H 4000, Clariant GmbH) are suspended in isopropanol. After the suspension has been rendered inert (evacuated and flooded with nitrogen), a solution of NaOH and water is run in. The mixture is rendered alkaline at 25° C. for 30 minutes. Then 3-(2,3-epoxypropoxy)propylphenyldimethylsilane is added as silylating reagent and the mixture is heated to 80° C. The reaction is left at this temperature for 4 hours. The mixture is then cooled at room temperature and neutralized with acetic acid against phenolphthalein. The product is filtered off with suction, washed salt-free with 85% strength isopropanol, digested again in 100% strength acetone, and dried in a drying oven at 70° C. For the batches a to f, Table 3 compiles the amounts of the reactants used, the degrees of substitution MS(OC₂H₄OH) and DS(organosilyl group), and the viscosities (2% strength solution, Hoeppler method) of the products. It is evident that the silylation brings about a marked increase in the viscosity.

TABLE 3
Batches a to f from Example 2

| | |
|---|---|
| Amounts used (g) | Degrees of |

| | | | |
|---|---|---|---|
| Tylose | Epoxyorgano- | substitution | Viscosities |

| Batch | H 4000 | Isopropanol | H₂O | NaOH | silane | MS | DS | (mPas) |
|---|---|---|---|---|---|---|---|---|
| a | 90 | 360 | 54 | 2.65 | — | 2.32 | — | 3770 |
| b | 90 | 360 | 54 | 2.65 | 1.65 | 2.32 | 0.007 | 4035 |
| c | 90 | 360 | 54 | 2.65 | 5.79 | 2.32 | 0.027 | 4941 |
| d | 90 | 360 | 54 | 2.65 | 8.28 | 2.32 | 0.036 | 5607 |
| e | 90 | 360 | 54 | 2.65 | 9.11 | 2.32 | 0.042 | 5459 |
| f | 90 | 360 | 54 | 2.65 | 9.19 | 2.32 | 0.064 | 6705 |

### Example 3

Preparation of an Inventive Cellulose Ether by Silylation of Hydroxyethylcellulose with 3-(2, 3-epoxypropoxy) propyltri-n-hexylsilane (Process (B))

The silylation of the hydroxyethylcellulose is carried out in analogy to the experimental procedure in Example 2. However, the silylating reagent used is 3-(2,3-epoxypropoxy)propyltri-n-hexylsilane. For the batches a to i, Table 4 compiles the amounts of the reactants used, the degrees of substitution MS(OC₂H₄OH) and DS(organosilyl group) and the viscosities (2% strength solution, Hoeppler method) of the products.

TABLE 4
Batches a to i from Example 3

| | |
|---|---|
| Amounts used (g) | Degrees of |

| | | | |
|---|---|---|---|
| Tylose | Epoxyorgano- | substitution | Viscosities |

| Batch | H 4000 | Isopropanol | H₂O | NaOH | silane | MS | DS | (mPas) |
|---|---|---|---|---|---|---|---|---|
| a | 90 | 360 | 54 | 2.65 | — | 2.32 | — | 3770 |
| b | 90 | 360 | 54 | 2.65 | 1.03 | 2.32 | 0.0004 | 4332 |
| c | 90 | 360 | 54 | 2.65 | 2.06 | 2.32 | 0.0007 | 6640 |
| d | 90 | 360 | 54 | 2.65 | 2.48 | 2.32 | 0.0009 | 7249 |
| e | 90 | 360 | 54 | 2.65 | 3.09 | 2.32 | 0.0011 | 16443 |
| f | 90 | 360 | 54 | 2.65 | 3.54 | 2.32 | 0.0015 | 27027 |
| g | 90 | 360 | 54 | 2.65 | 5.16 | 2.32 | 0.0019 | 28753 |
| h | 90 | 360 | 54 | 2.65 | 6.19 | 2.32 | 0.0022 | 124423 |
| i | 90 | 360 | 54 | 2.65 | 7.07 | 2.32 | 0.003 | 154550 |

### Example 4

Preparation of an Inventive Cellulose Ether by Silylation of hydroxyethylcellulose with 3-(2,3-epoxypropoxy)propyltriethylsilane (Process (B))

The silylation of hydroxyethylcellulose is carried out in analogy to the experimental procedure in Example 2. However, the silylating reagent used is 3-(2,3-epoxypropoxy)propyltriethylsilane. For the batches a to k, Table 5 compiles the amounts of the reactants used, the degrees of substitution MS(OC₂H₄OH) and DS(organosilyl group) and the viscosities (2% strength solution, Hoeppler method) of the products.

TABLE 5
Batches a to k from Example 4

| | |
|---|---|
| Amounts used (g) | Degrees of |

| | | | |
|---|---|---|---|
| Tylose | Epoxyorgano- | substitution | Viscosities |

| Batch | H 4000 | Isopropanol | H₂O | NaOH | silane | MS | DS | (mPas) |
|---|---|---|---|---|---|---|---|---|
| a | 90 | 360 | 54 | 2.65 | — | 2.32 | — | 3770 |
| b | 90 | 360 | 54 | 2.65 | 0.71 | 2.32 | 0.003 | 4681 |
| c | 90 | 360 | 54 | 2.65 | 1.44 | 2.32 | 0.004 | 5207 |
| d | 90 | 360 | 54 | 2.65 | 2.16 | 2.32 | 0.005 | 5379 |
| e | 90 | 360 | 54 | 2.65 | 2.88 | 2.32 | 0.008 | 5645 |
| f | 90 | 360 | 54 | 2.65 | 4.28 | 2.32 | 0.014 | 5759 |
| g | 90 | 360 | 54 | 2.65 | 5.13 | 2.32 | 0.011 | 5857 |
| h | 90 | 360 | 54 | 2.65 | 5.71 | 2.32 | 0.013 | 7080 |
| i | 90 | 360 | 54 | 2.65 | 6.60 | 2.32 | 0.020 | 10471 |
| j | 90 | 360 | 54 | 2.65 | 7.13 | 2.32 | 0.018 | 10475 |
| k | 90 | 360 | 54 | 2.65 | 8.56 | 2.32 | 0.026 | 28388 |

### Example 5

Preparation of an Inventive Cellulose Ether by Silylation of hydroxyethylcellulose with 3-(2,3-epoxypropoxy)propyloctadecyldimethylsilane (Process (B))

The silylation of hydroxyethylcellulose is carried out in analogy to the experimental procedure in Example 2. However, the silylating reagent used is 3-(2,3-epoxypropoxy)propyloctadecyldimethylsilane. For the batches a to p, Table 6 compiles the amounts of the reactants used, the degrees of substitution MS(OC₂H₄OH) and DS(organosilyl group) and the viscosities (2% strength solution, Hoeppler method) of the products.

TABLE 6
Batches a to p from Example 5

| | |
|---|---|
| Amounts used (g) | Degrees of |

| | | | |
|---|---|---|---|
| Tylose | Epoxyorgano- | substitution | Viscosities |

| Batch | H 4000 | Isopropanol | H₂O | NaOH | silane | MS | DS | (mPas) |
|---|---|---|---|---|---|---|---|---|
| a | 90 | 360 | 54 | 2.65 | — | 2.32 | — | 3770 |
| b | 90 | 360 | 54 | 2.65 | 0.14 | 2.32 | <0.0025 | 5255 |
| c | 90 | 360 | 54 | 2.65 | 0.41 | 2.32 | <0.0025 | 4947 |
| d | 90 | 360 | 54 | 2.65 | 0.55 | 2.32 | <0.0025 | 7757 |
| e | 90 | 360 | 54 | 2.65 | 0.69 | 2.32 | <0.0025 | 11663 |
| f | 90 | 360 | 54 | 2.65 | 0.97 | 2.32 | <0.0025 | 15745 |
| g | 90 | 360 | 54 | 2.65 | 1.24 | 2.32 | <0.0025 | 34217 |
| h | 90 | 360 | 54 | 2.65 | 1.38 | 2.32 | <0.0025 | 36453 |
| i | 90 | 360 | 54 | 2.65 | 1.66 | 2.32 | <0.0025 | 72333 |
| j | 90 | 360 | 54 | 2.65 | 1.79 | 2.32 | <0.0025 | 117014 |
| k | 90 | 360 | 54 | 2.65 | 2.07 | 2.32 | <0.0025 | 181622 |
| l | 90 | 360 | 54 | 2.65 | 2.76 | 2.32 | <0.0025 | 477990 |
| m | 90 | 360 | 54 | 2.65 | 3.45 | 2.32 | <0.0025 | Gel |
| n | 90 | 360 | 54 | 2.65 | 4.14 | 2.32 | <0.0025 | Gel |
| o | 90 | 360 | 54 | 2.65 | 6.92 | 2.32 | 0.0025 | Gel |
| p | 90 | 360 | 54 | 2.65 | 8.29 | 2.32 | 0.0027 | Gel |

1. A water-soluble cellulose ether silylated with at least one organosilyl group containing one or more silicon atoms, wherein the organosilyl group(s) is (are) attached hydrolysis-stably to the cellulose ether and the remaining substituents of the silicon atom or silicon atoms of the organosilyl group(s) are stable to hydrolysis.

2. The cellulose ether as claimed in claim 1, wherein the silyl-containing reagent or reagents for silylating the cellulose ether preferably comprise organosilicon compounds having the compositions 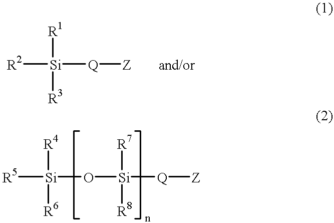
where
a) R¹ to R⁸ independently of one another are identical or different and are straight-chain or branched alkyl radicals, straight-chain or branched alkenyl radicals, aryl radicals and/or arylalkyl radicals containing straight-chain and/or branched alkyl groups,
b) Z is a reactive functional group suitable for covalent bonding to cellulose ethers and selected from the group consisting of Cl, Br, I, isocyanate, epoxy groups, glycidyloxy groups, acid halide groups and/or acid anhydride groups,
c) Q is a spacer group between the reactive group Z and the organosilyl group, and
d) n is from 1 to 100.

3. The cellulose ether as claimed in claim 2, wherein R¹ to R⁸ are C₁-C₂₀ alkyl radicals and/or phenyl radicals, Z is an epoxy group, and Q is a C₁-C₂₀ hydrocarbon chain.

4. The cellulose ether as claimed in claim 1, wherein the average number of silyl groups per anhydroglucose unit (DS silyl) is from 0.0002 to 0.2.

5. The cellulose ether as claimed in claim 1, wherein the cellulose ether comprises a silylated hydroxyethylcellulose, a silylated hydroxypropylcellulose, a silylated carboxymethylcellulose, a silylated carboxymethylhydroxyethylcellulose, a silylated methylcellulose, a silylated methylhydroxyethyl cellulose or a silylated methylhydroxypropylcellulose.

6. A process for preparing a silylated cellulose ether as claimed in claim 1, which comprises reacting cellulose with base catalysis with one or more etherifying agents selected from the group consisting of ethylene oxide, propylene oxide, methyl chloride, monochloroacetic acid and sodium monochloroacetate and with at least one silylating reagent.

7. A process for preparing a cellulose ether as claimed in claim 1, which comprises reacting a cellulose ether with base catalysis with at least one silylating reagent.

8. The process as claimed in claim 7, wherein the cellulose ether to be silylated comprises hydroxyethylcellulose, hydroxypropylcellulose, carboxymethylcellulose, carboxymethylhydroxyethylcellulose, methylcellulose, methylhydroxyethylcellulose or methylhydroxypropylcellulose.

9. The process for preparing a silylated cellulose ether which comprises reacting cellulose with base catalysis with one or more etherifying agents selected from the group consisting of ethylene oxide, propylene oxide, methyl chloride, monochloroacetic acid and sodium monochloroacetate and with at least one silylating reagent, wherein the silylating reagents comprise organosilicon compounds as set forth in claim 2.

10. A method for the preparation of paints, adhesives or cosmetics, wherein a silylated cellulose ether or of a mixture of silylated cellulose ethers as claimed in claim 1 is used as a thickening auxiliary.

11. A method for the preparation of polymer dispersions, wherein a silylated cellulose ether or of a mixture of silylated cellulose ethers as claimed in claim 1 is used as a protective colloid.

12. A process for preparing a cellulose ether which comprises reacting a cellulose ether with base catalysis with at least one silylating reagent, wherein the silylating reagents comprise organosilicon compounds as set forth in claim 2.
